(19) United States
(12) Patent Application Publication
(10) Pub. No.: US 2006/0251745 A1
(43) Pub. Date: Nov. 9, 2006

(54) Method for preparing a Ginkgo extract having a reduced content of 4'-O-methyl pyridoxine and/or biflavones

(75) Inventors: Clemens Erdelmeier, Karlsruhe (DE); Egon Koch, Karlsruhe (DE); Karl-Heinz Stumpf, Karlsruhe (DE); Hermann Hauer, Karlsruhe (DE); Friedrich Lang, Hagenbach (DE)

Correspondence Address: EDWARDS & ANGELL, LLP, P.O. BOX 55874, BOSTON, MA, 02205, US

(73) Assignee: Bioplanta Arzneimittel GmbH, Ettlingen (DE)

(21) Appl. No.: 11/416,878

(22) Filed: May 2, 2006

(30) Foreign Application Priority Data
May 3, 2005 (DE) DE 102005020642.5

(51) Int. Cl. A61K 36/16

(52) U.S. Cl. 424/752

(57) ABSTRACT

The present invention relates to a method for preparing an extract from Ginkgo biloba having a reduced content of 4′-O-methyl pyridoxine and/or biflavones compared to the original extract.

The invention further relates to extracts from Ginkgo biloba having a reduced content of 4′-O-methyl pyridoxine and/or biflavones compared to the original extract, which is obtainable by the method according to the present invention, as well as to their use.

The present invention relates to a method for preparing an extract from Ginkgo biloba having a reduced content of 4′-O-methyl pyridoxine and/or biflavones compared to the original extract, characterized by the following steps of the method:
(a) preparing a Ginkgo extract solution in a suitable solvent and
(b) applying the solution to an adsorber resin and eluting the purified extract from the adsorber resin using a suitable solvent, wherein the biflavones to be removed remain on the adsorber resin, and/or
(c) applying the solution to an acidic ion exchanger and eluting the purified extract from the ion exchanger using a suitable solvent, wherein the 4′-O-methyl pyridoxine to be removed remains on the ion exchanger, and optionally,
(d) concentrating and drying the extract solution to obtain a dry extract,
   wherein steps (b) and (c) may also be carried out in reverse order.

The present invention further relates to extracts from Ginkgo biloba having a reduced content of 4′-O-methyl pyridoxine and/or biflavones compared to the original extract, the extracts from Ginkgo biloba being obtainable by the method according to the present invention, as well as to their use.

Since decades, extracts from the leaves of Ginkgo biloba are used as a medicament. They are currently used for the treatment of different types of dementia and symptoms thereof as well as cerebral and peripheral blood circulation disorders. Ingredients, the efficacy is associated with, are terpene lactones (ginkgolides A, B, and C und bilobalide) as well as glycosides of flavones (quercetin, kaempferol and isorhamnetin). However, the leaves of Ginkgo biloba also contain components, which do not contribute to the desired efficacy, but which may be responsible for risks and side effects. In addition to unpolar plant ingredients, such as ginkgolic acids, those components are 4′-O-methyl pyridoxine and biflavones. In a Ginkgo extract, which is efficacious and at the same time as safe as possible and as low in side effects as possible, these compounds should thus not be present to the largest possible extent. 4′-O-methyl pyridoxine may cause symptoms of poisoning such as convulsive seizures and unconsciousness. Thus, this compound is also referred to as ginkgotoxin. The biflavones contained in ginkgo exhibit an immunotoxic potential and may elicit contact allergies. These biflavones contained in ginkgo are predominantly the compounds amentoflavone, bilobetin, ginkgetin, isoginkgetin and sciadopitysin.

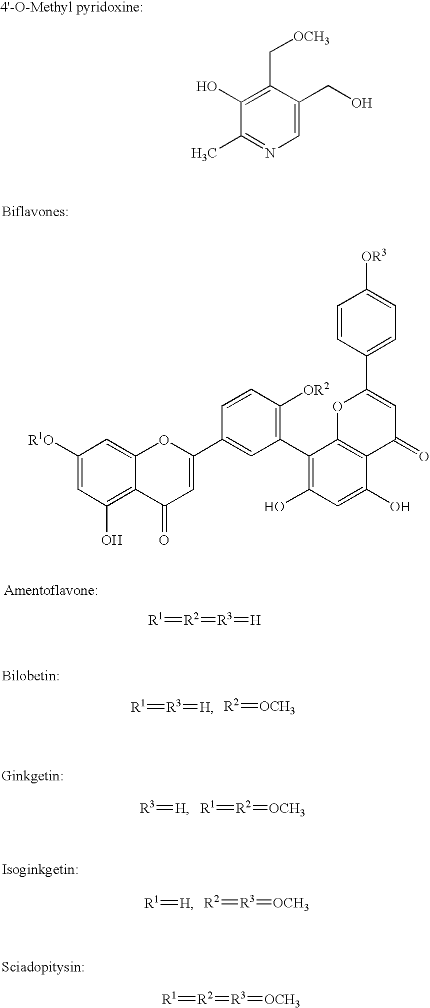

Methods for depleting 4′-O-methyl pyridoxine and biflavones as well as the extracts obtained are already described in EP 1 037 646 B1. Therein, 4′-O-methyl pyridoxine is retained using an acidic cation exchanger and the biflavones are adsorbed on activated carbon. Preferably these depletion steps are carried out after step f) of the method according to EP 1 037 646 B1 (page 3), i.e., after “treating the solution with a lead compound or an insoluble polyamide”.

In EP 1 037 646 B1 the depletion of the biflavones and 4′-O-methyl pyridoxine is carried out at a stage of the method (step f)), wherein relatively high contents of lead salts and ammonium salts are present such that an increased amount of ion exchanger has to be employed.

However, this method is limited to a complex sequence of several work-up steps.

Furthermore, the depletions of biflavones and 4′-O-methyl pyridoxine are described and claimed in combination only. However, depending on the limits to be possibly established in the future and in view of the requirement of as low a modification of the extract composition as possible, it may be desirable to remove the biflavones or 4′-O-methyl pyridoxine only.

Moreover, the depletion is merely described with one single ion exchanger (Merck I) in the case of 4′-O-methyl pyridoxine and with one single adsorbent (active carbon) in the case of biflavones. For this purpose, active carbon has the drawback that it typically does not bind in a very selective manner and cannot be regenerated.

Thus, it is the object underlying the present invention to provide a method for preparing Ginkgo extracts, wherein 4′-O-methyl pyridoxine and/or biflavones can be depleted to the largest extent possible compared to the original extract and which can additionally be carried out in a simple and cost-efficient manner. A further subject of the present invention are Ginkgo extracts, which are obtainable according to this method.

This object could be solved by separating the biflavones by adsorber resins and/or separating 4′-O-methyl pyridoxine via acidic ion exchangers.

In the method according to the present invention, the following method steps are performed:
(a) preparing a Ginkgo extract solution in a suitable solvent, as well as
(b) applying the solution to an adsorber resin and eluting the purified extract from the adsorber resin using a suitable solvent, wherein the biflavones to be removed remain on the adsorber resin and may be eluted from the resin using organic solvents, such as acetone, so that the resin can be employed again, and/or
(c) applying the solution to an acidic ion exchanger and eluting the purified extract from the ion exchanger using a suitable solvent, wherein the 4′-O-methyl pyridoxine to be removed remains on the ion exchanger and may be washed from the ion exchanger using an acid such as aqueous hydrochloric acid, so that the ion exchanger can be employed again, and optionally
(d) concentrating and drying the extract solution to obtain a dry extract, wherein steps (b) and (c) may also be carried out in reverse order.

Particularly suitable solvents in steps (a), (b) and (c) are independently aqueous ketones having 3 to 6 carbon atoms (such as acetone, 2-butanon) and alkanols having 1 to 3 carbon atoms (methanol, ethanol, n-propanol and isopropanol), preferably aqueous acetone and ethanol, a concentration of 30 to 60% by weight being preferred. Preferred adsorber resins in step (b) are resins on the basis of optionally substituted styrenes/divinylbenzenes such as Diaion HP-20, HP-21 or Sepabeads SP-207 and SP-850. Particularly preferred adsorber resins are copolymers on the basis of styrene and divinylbenzene and copolymers on the basis of brominated styrene and divinylbenzene. Preferred ion exchangers in step (c) are strongly acidic ion exchangers such as Merck I or Amberlite IR-120.

The original extract used to determine the original value is obtained from the original solution (Ginkgo extract solution according to step (a)) by drying to the dry extract.

In step (b) of the method according to the present invention, the amount of the resin used as well as the polarity or the composition of the solvent used have to be adjusted such that the desired components of the extract are eluted from the resin to the largest possible extent first, whereas the biflavones remain on the resin. Thereby, a higher content of water in the solvent leads to a better depletion of the biflavones, i.e., the biflavones remain on the resin. Depending on the solubility of the extract, the water content of the solvent is within the above-mentioned range of 30 to 70% by weight.

As original extracts (in the form of Ginkgo extract solutions according to step (a) or obtained therefrom) which are to be set free from 4′-O-methyl pyridoxine and/or biflavones by means of the adsorber resin, the following can be considered:
single-fold extracts prepared according to methods known per se, for example, according to the European Pharmacopoeia, by extracting dried leaves of Ginkgo biloba using organic solvents or mixtures thereof with water, for example, using ethanol/water mixtures or acetone/water mixtures or
extracts, the composition of which differs from the underlying single-fold extract due to one or more additional method steps to a greater or lesser extent (so-called special extracts).

The latter extracts can be prepared, for example, by a liquid-liquid distribution according to EP 360556 B1 or by some steps according to EP 431535 B1 such as steps (a) to (c) or by the entire method according to EP 431535 B1 or according to U.S. Pat. No. 6,117,431.

The Ginkgo extract solutions according to step (a) can either be obtained directly from the preparation process of the extract or by dissolving a dry extract or another extract.

A further subject of the present invention are extracts, in particular dry extracts which are obtainable by the method of the present invention and which are characterized by a reduced content of 4′-O-methyl pyridoxine and/or biflavones compared to the employed original extracts. The contents of 4′-O-methyl pyridoxine are 20 ppm at the most in the case of extracts according to the present invention, preferably 10 ppm at the most, and in particular 5 ppm at the most. The contents of biflavones are 25% of the original value of the original extract at the most, preferably 11% at the most, and in particular 6% at the most.

According to the European Pharmacopoeia dry extracts generally have a dry residue of at least 95% by weight.

The extracts according to the present invention can be administered in the form of powders, granules, tablets, dragees (coated tablets) or capsules, preferably orally. In order to prepare tablets, the extract is mixed with suitable pharmaceutically acceptable adjuvants, such as lactose, cellulose, silicon dioxide, croscarmellose and magnesium stearate and pressed into tablets which are optionally provided with a suitable coating made of, for example, hydroxymethylcellulose, polyethyleneglycol, pigments (such as titanium dioxide, iron oxide) and talcum. The extract according to the present invention can also be filled into capsules, optionally under the addition of adjuvants such as stabilizers, fillers and the like. The dosis is such that 1 of 2000 mg, preferably 50-1000 mg, and particularly preferred 100-500 mg of the extract are administered per day.

Moreover, subject of the present invention are medicaments, food products or other preparations which contain these extracts optionally in combination with other substances such as active ingredients and/or adjuvants. The term “food product” as used herein particularly refers to dietetic food products, dietary supplement products as well as medical food and dietary supplements.

### EXAMPLES

### Original Solution For Comparative Example 1 And Examples 1 To 3 According To the Present Invention

200 g dried and ground leaves of Ginkgo biloba were extracted twice using each time 7 times their weight (w/w) made up of ethanol/water 60/40 (w/w) at a temperature of about 50° C. and filtered.

### Comparative Example 1

### Preparation of A Single-Fold Extract As the Original Extract

25% of the original solution obtained above were concentrated under reduced pressure and freeze-dried: 14.0 g (28.0% based on the dried leaves).

### Example 1

### According To the Invention (Removal of Biflavones)

25% of the original solution obtained above were applied to a column with 100 ml Sepabeads SP-850 adsorber resin and eluted with 300 ml 40% by weight ethanol. The eluate was concentrated under reduced pressure and freeze-dried: 11.5 g (23.0% based on the dried leaves).

### Example 2

### According To the Invention (Removal of 4′-O-Methyl Pyridoxine)

25% of the original solution obtained above were applied to a column with 20 ml strongly acidic ion exchanger Amberlite IR-120 and eluted with 60 ml 60% by weight ethanol. The eluate was concentrated under reduced pressure and freeze-dried: 13.0 g (26.0% based on the dried leaves).

### Example 3

### According To the Invention (Removal of Biflavones And 4′-O-Methyl Pyridoxine)

25% of the original solution obtained above were applied to a first column with 100 ml Sepabeads SP-850 adsorber resin and eluted with 300 ml 40% by weight ethanol.

The eluate was applied to a second column with 20 ml strongly acidic ion exchanger Amberlite IR-120 and eluted with 100 ml 40% by weight ethanol. The resulting solution was concentrated under reduced pressure and freeze-dried: 10.1 g (20.2% based on the dried leaves).

### Original Solution For Comparative Example 2 And Examples 4 To 6 According To the Present Invention

200g dried and ground leaves of Ginkgo biloba were extracted twice using each time seven times their weight (w/w) made of ethanol/water 60/40 (w/w) at a temperature of about 50° C. and filtered. The extract solution was largely concentrated (145 g), diluted with water to about 400 g and stored for about 19 h at 10° C. The precipitate formed was removed by filtration.

### Comparative Example 2

### Preparation of A Special Extract As the Original Extract

25% of the original solution obtained above were concentrated under reduced pressure and freeze-dried: 9.4 g (18.8% based on the dried leaves).

### Example 4

### According To the Present Invention (Removal of Biflavones)

25% of the original solution obtained above were applied to a column with 50 ml Sepabeads SP-207 adsorber resin and eluted with 150 ml 40% by weight ethanol. The eluate was concentrated under reduced pressure and freeze-dried: 9.4 g (18.8% based on the dried leaves).

### Example 5

### According To the Present Invention (Removal of 4′-O-Methyl Pyridoxine)

25% of the original solution obtained above were applied to a column with 20 ml strongly acidic ion exchanger Amberlite IR-120 and eluted with 100 ml 40% by weight ethanol. The eluate was concentrated under reduced pressure and freeze-dried: 8.7 g (17.4% based on the dried leaves).

### Example 6

### According To the Present Invention (Removal of Biflavones And 4′-O-Methyl Pyridoxine)

25% of the original solution obtained above were applied to a first column with 50 ml Sepabeads SP-207 adsorber resin and eluted with 150 ml 40% by weight ethanol. The eluate was applied to a second column with 20 ml strongly acidic ion exchanger Amberlite IR-120 and eluted with 100 ml 40% by weight ethanol. The resulting solution was concentrated under reduced pressure and freeze-dried: 8.3 g (16.6% based on the dried leaves).

The biflavone contents and the 4′-O-methyl pyridoxine contents of the resulting extracts can be taken from the following table. It is to be seen that the sum of the biflavones in Examples 1, 3, 4 and 6 according to the present invention is significantly lower than in the corresponding Comparative Examples 1 and 2, respectively. Analogously, the contents of 4′-O-methyl pyridoxine are significantly reduced in Examples 2, 3, 5 and 6 according to the present invention compared to the corresponding Comparative Examples 1 and 2, respectively.

TABLE 1
Composition of the extracts according to the above examples

| | Extract according to | | | |
|---|---|---|---|---|
| | Comparative | Example according to the | | |
| | Example | invention | | |
| | 1 | 1 | 2 | 3 |
| 4′-O-Methyl pyridoxine | 23 | 40 | 2 | 6 |
| [ppm] | | | | |
| Amentoflavone [ppm] | 72 | 112 | 92 | 107 |
| Bilobetin [ppm] | 780 | 530 | 1031 | 593 |
| Ginkgetin [ppm] | 2303 | 91 | 2792 | 143 |
| Isoginkgetin [ppm] | 2877 | 139 | 3418 | 208 |
| Sciadopitysin [ppm] | 4524 | 2 | 5400 | 6 |
| Total amount of biflavones | 10556 | 874 | 12733 | 1057 |
| [ppm] | | | | |

| | Extract according to | | | |
|---|---|---|---|---|
| | Comparative | Example according to the | | |
| | Example | invention | | |
| | 2 | 4 | 5 | 6 |
| 4′-O-Methyl pyridoxine | 29 | 29 | <1 | <1 |
| [ppm] | | | | |
| Amentoflavone [ppm] | 7 | <0.1 | 6 | approx. |
| | | | | 2 |
| Bilobetin [ppm] | 20 | approx. | 16 | approx. |
| | | 0.1 | | 1 |
| Ginkgetin [ppm] | 17 | approx. | 14 | approx. |
| | | 0.2 | | 0.7 |
| Isoginkgetin [ppm] | 23 | approx.. | 16 | approx. |
| | | 0.3 | | 0.6 |
| Sciadopitysin [ppm] | 22 | approx. | 16 | approx. |
| | | 0.4 | | 0.6 |
| Total amount of biflavones | 89 | approx. | 68 | approx. |
| [ppm] | | 1 | | 5 |

1-17. (canceled)

18. A method for preparing an extract from Ginkgo biloba having a reduced content of 4′-O-methyl pyridoxine and/or biflavones compared to the original extract, the method comprising:
(a) preparing a Ginkgo extract solution in a solvent,
and
(b) applying the solution to an adsorber resin and eluting the purified extract from the adsorber resin using a solvent, wherein the biflavones to be removed remain on the adsorber resin,
and/or
(c) applying the solution to an acidic ion exchanger and eluting the purified extract from the ion exchanger using a solvent, wherein the 4′-O-methyl pyridoxine to be removed remains on the ion exchanger,
and optionally
(d) concentrating and drying the extract solution to the dry extract,
wherein steps (b) and (c) can also be carried out in reverse order.

19. A method of claim 18 wherein the solvent in steps (a), (b) and (c) is independently selected from an aqueous alkanol having 1 to 3 carbon atoms and an aqueous ketone having 3 to 6 carbon atoms.

20. A method of claim 19 wherein the alkanol is methanol, ethanol, n-propanol or isopropanol and the ketone is acetone.

21. A method of claim 19 wherein the water content is in a range from 30 to 70% by weight.

22. A method of claim 19 wherein the water content of the solvent in steps (a), (b) and (c) is equal or different.

23. A method of claim 18 wherein the adsorber resin is a copolymer comprising styrene and divinylbenzene.

24. A method of claim 18 wherein the adsorber resin is a copolymer comprising brominated styrene and divinylbenzene.

25. A method of claim 18 wherein the ion exchanger is a strongly acidic ion exchanger.

26. An extract from Ginkgo biloba having a reduced content of 4′-O-methyl pyridoxine and/or biflavones compared to the original extract, which is obtainable according to the method of claim 18.

27. An extract of claim 26 wherein the content of 4′-O-methyl pyridoxine is 20 ppm at the most.

28. An extract of claim 26 wherein the content of 4′-O-methyl pyridoxine is 10 ppm at the most.

29. An extract of claim 26 wherein the content of 4′-O-methyl pyridoxine is 5 ppm at the most.

30. An extract of claim 26 wherein the content of biflavones is 25% of the original value of the original extract at the most.

31. An extract of claim 26 wherein the content of biflavones is 11% of the original value of the original extract at the most.

32. An extract of claim 26 wherein the content of biflavones is 6% of the original value of the original extract at the most.

33. A method of treating a subject suffering from or susceptible to dementia and one or more symptoms thereof and/or a cerebral or peripheral blood circulation disorder, comprising administering to the subject an extract of claim 26.

34. A method of claim 33 wherein the subject is suffering from dementia and one or more symptoms thereof and/or a cerebral or peripheral blood circulation disorder.

35. A method of claim 33 wherein the subject is suffering from dementia.

36. A method of claim 33 wherein the subject is suffering from a cerebral or peripheral blood circulation disorder.

37. A method of claim 33 wherein the subject is identified as suffering from dementia and one or more symptoms thereof and/or a cerebral or peripheral blood circulation disorder and the extract is administered to the identified subject.

38. A medicament, food product or other preparation comprising a Gingko extract of claim 26.

39. A method for preparing an extract from Ginkgo biloba having a reduced content of 4′-O-methyl pyridoxine and/or biflavones compared to the original extract, compsing:
(a) preparing a Ginkgo extract solution in a solvent,
and
(b) applying the solution to an adsorber resin and eluting the purified extract from the adsorber resin, wherein the biflavones to be removed remain on the adsorber resin,
and/or
(c) applying the solution to an acidic ion exchanger and eluting the purified extract from the ion exchanger, wherein the 4′-O-methyl pyridoxine to be removed remains on the ion exchanger,
wherein if both steps (b) and (c) are conducted, step (b) can be conducted prior to step (c), or step (c) may be conducted prior to step (b).

40. A method of claim 39 further comprising concentrating and drying the extract solution.
